(19) United States
(12) Patent Application Publication
(10) Pub. No.: US 2026/0287565 A1
(43) Pub. Date: Sep. 24, 2026

(54) METHOD OF MASS SPECTROMETRY FOR THE DETECTION OF PERFLUOROALKYL OR POLYFLUOROALKYL SUBSTANCES (PFAS)

(71) Applicant: Thermo Fisher Scientific (Bremen) GmbH, Bremen (DE)

(72) Inventor: Ralf Hartmer, Bremen (DE)

(73) Assignee: Thermo Fisher Scientific (Bremen) GmbH, Bremen (DE)

(21) Appl. No.: 19/473,793

(22) PCT Filed: Mar. 28, 2024
(86) PCT No.: PCT/EP2024/058567

§ 371 Date: Oct. 8, 2025

(30) Foreign Application Priority Data
Apr. 11, 2023 (GB) 2305277.2

(51) Int. Cl. G01N 30/72; B01D 15/08; B01D 53/02; G01N 30/02; G01N 30/86; H01J 49/14; H01J 49/42

(52) U.S. Cl. CPC G01N 30/7266; B01D 15/08; B01D 53/025; G01N 30/8631; H01J 49/147; H01J 49/425; G01N 2030/027

(57) ABSTRACT

A method of determining the presence of a perfluoro or polyfluoro compound (PFC), such as a PFAS compound, comprises generating sample ions from a sample using a mass analyser to produce mass spectral data. The mass analyser has a mass accuracy ≤about 100 ppm and a resolving power ≥about 10,000. The method further comprises determining whether, in the mass spectral data, an ion peak is present that has an accurate mass to charge ratio within ±100 ppm of a first accurate mass to charge ratio m/z indicative of the presence of a PFC in the sample. The method further comprises determining that a PFC is present in the sample when it is determined that an ion peak is present that has an accurate mass to charge ratio within ±100 ppm of the first accurate mass to charge ratio m/z.

### FIELD OF THE INVENTION

The present invention relates to the field of mass spectrometry, and in particular to mass spectrometric methods of detecting per- and polyfluoro compounds (PFCs), in particular, but not exclusively, per- and polyfluoroalkyl substances (PFAS).

### BACKGROUND

Per- and poly-fluoroalkyl substances (PFAS) are a group of synthetic chemicals that have been used in many products such as firefighting foam, chrome-plating, waterproof textiles, and other products. They have been widely used in industry and manufacturing due to their unique properties, including being heat-resistant, able to repel water, and highly robust. However, these chemicals are currently attracting particular attention due to their persistence in the environment and risk of adverse health impacts.

More than 4700 identified PFAS substances are known. PFAS comprise a carbon chain, in particular an alkyl chain, in which hydrogen atoms are entirely or partly replaced by fluorine atoms. Thus, PFAS have multiple carbon-fluorine bonds. A detailed list of highly fluorinated compounds is given in the KEMI 7/15 report, Occurrence and use of highly fluorinated substances and alternatives, covering 2060 identified substances with a CAS number and an estimation of substances without a CAS number.

There is a great need for the identification and accessible screening of PFAS and PFC. In 2021, the EPA (US) environmental protection agency released a PFAS strategic roadmap as a whole-of-agency approach addressing the impact of PFAS on the environment.

Currently, non-mass spectrometry-based techniques are most commonly utilised for screening of PFAS and PFC. For example, in CN113791057 describes a perfluorinated compound high-throughput screening method using a fluorescence sensor array, WO2021/142455 describes screening/analysis of fluorocarbons using x-ray photoelectron spectroscopy, and U.S. Pat. No. 11,002,691 describes a method for detecting fluorinated chemicals in liquid. These non-MS techniques have the risk of either not being sensitive enough or providing false positive results because of the low specificity of the technique.

Other known methods use a targeted mass spectrometric approach. For example, CN105784881 describes a method of determining the presence of perfluorinated compound isomers in soil and/or plants, and CN105467026 describes a method for detecting perfluorinated compounds in soil and sediments. However, these methods do not provide a general PFAS screening approach.

WO202183894 describes a method of parallel analysis of a volatilized sample under two different ionization conditions, enabling simultaneous characterisation of a chromatographically separated compound under two different ionisation condition such as harsh ionisation by electron impact (EI) or soft ionisation by chemical ionisation (CI).

It is believed that there remains scope for improvements to apparatus and methods for mass analysis.

### SUMMARY

A first aspect provides a method of determining the presence of a perfluoro or polyfluoro compound (PFC) in a sample, the method comprising:
generating sample ions from a sample either: (i) by using electron impact (EI) ionisation, direct laser ionisation, or field desorption ionisation; or (ii) by using electrospray ionisation (ESI) or chemical ionisation (CI), and electron induced dissociation (EID);
mass analysing the sample ions using a mass analyser so as to produce mass spectral data, wherein the mass analyser has a mass accuracy ≤about 100 ppm and a resolving power ≥about 10,000;
determining whether, in the mass spectral data, an ion peak is present that has an accurate mass to charge ratio within ±100 ppm of a first accurate mass to charge ratio m/z, wherein the first accurate mass to charge ratio m/z is the accurate mass to charge ratio of an ion indicative of the presence of a PFC in the sample; and
when it is determined that an ion peak is present that has an accurate mass to charge ratio within ±100 ppm of the first accurate mass to charge ratio m/z: determining that a PFC is present in the sample.

The PFC can typically be a PFAS compound. Existing mass spectrometric methods of identifying PFAS compounds use library searches, but these methods suffer from the incompleteness of libraries as there are many thousands of different PFAS compounds. Furthermore, existing methods commonly use soft ionisation techniques such as electrospray ionisation (ESI), which do not produce ions universally indicative of the presence of a PFAS, such as the CF3 cation.

According to embodiments, electron impact (EI) ionisation or other similar “harsh” ionisation techniques are specifically used to produce ions universally indicative of the presence of a PFAS compound, such as the CF3 cation. Alternatively, a soft ionisation technique such as electrospray ionisation (ESI) or chemical ionisation (CI) is used together with electron induced dissociation (EID) to produce ions universally indicative of the presence of a PFAS compound, such as the CF3 cation. As described further below, these ions have a slightly lower accurate mass than their nominal mass, which allows them to be distinguished from other ions with the same nominal mass when a sufficiently high mass accuracy, high resolution mass analyser (such as an electrostatic orbital ion trap mass analyser) is used.

The sample ions may be generated from the sample by ionising the sample using electron impact (EI) ionisation, direct laser ionisation, or field desorption ionisation. In particular, the sample ions may be generated from the sample using electron impact (EI) ionisation.

Alternatively, the sample ions may be generated from the sample by ionising the sample using electrospray ionisation (ESI) or chemical ionisation (CI) to produce parent ions, and then fragmenting the parent ions using electron induced dissociation (EID) so as to produce the sample ions. In these embodiments, the electrospray ionisation (ESI) or chemical ionisation (CI) may be operated in a negative ionisation mode.

The mass analyser may be an electrostatic orbital ion trap mass analyser, a Time-of-Flight (ToF) mass analyser, a multi-reflection Time-of-Flight (mrTOF) mass analyser, or a Fourier transform ion cyclotron resonance (FT-ICR) mass analyser.

The mass analyser may be an electrostatic orbital ion trap mass analyser.

Where the PFC is a PFAS, the ion indicative of the presence of a PFC is an ion indicative of the presence of a PFAS in the sample.

The ion indicative of the presence of a PFAS may be a CF3 cation, wherein the first accurate mass to charge ratio m/z=68.99466.

The ion indicative of the presence of a PFAS may be a CF3CF2 cation, wherein the first accurate mass to charge ratio m/z=118.99147.

The ion indicative of the presence of a PFAS may be a CF3CF cation, wherein the first accurate mass to charge ratio m/z=130.99147.

The method may further comprise, before the step of mass analysing the sample ions: isolating sample ions having mass to charge ratios within a mass to charge ratio window centred at the nominal mass to charge ratio of the ion indicative of the presence of a PFC or PFAS.

The step of isolating sample ions may comprise isolating sample ions using a mass filter such as a quadrupole mass filter.

The mass to charge ratio window may have a width ≤10 m/z, or ≤5 m/z.

The ion indicative of the presence of a PFAS may be a CF3 cation, wherein the nominal mass to charge ratio is 69.

The ion indicative of the presence of a PFAS may be a CF3CF2 cation, wherein the nominal mass to charge ratio is 119.

The ion indicative of the presence of a PFAS may be a CF3CF cation, wherein the nominal mass to charge ratio is 131.

The step of determining whether an ion peak is present may comprise determining whether an ion peak is present that has an intensity above a signal to noise ratio threshold. The signal to noise ratio threshold may be about 50.

The method may further comprise chromatographically separating the sample before the step of generating sample ions from the sample.

The step of chromatographically separating the sample may comprise separating the sample using liquid chromatography (LC) or gas chromatography (GC).

The method may further comprise:
splitting the chromatographically separated sample into a first part and a second part, wherein the sample ions are first sample ions generated by ionising the first part of the sample using a first ion source, wherein the mass spectral data is first mass spectral data, and wherein the method further comprises:
generating second sample ions from the second part of the sample using a second different ion source; and
mass analysing the second sample ions so as to produce second mass spectral data.

Splitting the chromatographically separated sample into the first part and the second part may comprise continuously splitting the chromatographically separated sample into the first part and the second part. In these embodiments, the first sample ions and the second sample ions may be generated simultaneously.

Alternatively, splitting the chromatographically separated sample into the first part and the second part may comprise: alternately performing the following steps (i) and (ii): (i) directing the chromatographically separated sample to the first ion source; and then (ii) directing the chromatographically separated sample to the second ion source. In these embodiments, generation of the first sample ions and the second sample ions may be interleaved in time (i.e. the first sample ions and the second sample ions are not generated simultaneously).

The second ion source may be an electrospray (ESI) ion source or a chemical ionisation (CI) ion source.

The first sample ions and the second sample ions may be mass analysed using the same mass analyser. In these embodiments, mass analysis of the first sample ions and the second sample ions may be interleaved in time.

The first sample ions may be mass analysed using a first mass analyser, and the second sample ions may be mass analysed using a second different mass analyser. In these embodiments, mass analysis of the first sample ions and the second sample ions may be either simultaneous or interleaved in time.

The method may further comprise:
when it is determined that an ion peak is present that has an accurate mass to charge ratio within ±100 ppm of the first accurate mass to charge ratio m/z: determining a chromatographic retention time associated with the ion peak; and
characterising or identifying the PFC or PFAS present in the sample using the second mass spectral data at or approximately at (e.g. closest in time to) the determined chromatographic retention time.

The method may further comprise searching a library of spectral data of PFC or PFAS compounds to characterise or identify the PFC or PFAS present in the sample. The method may further comprise comparing the second mass spectral data to spectral data in the library to find matching data in the library which characterises or identifies the PFC or PFAS.

The sample may be an environmental sample, such as a water sample or a soil sample, optionally dissolved in a solvent.

The method may further comprise: when it is determined that an ion peak that has an accurate mass to charge ratio within ±100 ppm of the first accurate mass to charge ratio m/z is not present in the mass spectral data: determining that a PFC or PFAS is not present in the sample. It may be determined an ion peak is not present when an ion peak is not present, or when an ion peak is present that has an intensity below the signal to noise ratio threshold.

The method may further comprise adding a known concentration of labelled sample to the non-labelled sample, such that the sample ions include sample ions derived from the non-labelled sample and sample ions derived from the labelled sample. The labelled sample may comprise, for example, C13 enriched labelled sample. The step of generating sample ions (when using a “harsh” ionisation source) may produce sample fragment ions (i.e. fragment ions derived from the non-labelled sample) and labelled sample fragment ions (i.e. fragment ions derived from the labelled sample). Alternatively, where a “soft” ionisation source is used (as described above), the method may further comprise fragmenting the ions produced by the second ion source to produce sample fragment ions (i.e. fragment ions derived from the non-labelled sample) and labelled sample fragment ions (i.e. fragment ions derived from the labelled sample). In either case, the fragment ions may be mass analysed to produce mass spectral data of the sample fragment ions and the labelled sample fragment ions. One or more pairs of corresponding fragment ions may be identified in the mass spectral data (with each pair comprising a non-labelled fragment ion and its corresponding C13 enriched labelled fragment ion), and the relative intensity of each pair of corresponding fragment ions may be determined. The relative intensity or intensities may be used to quantify the concentration of PFC present in the sample.

A further aspect provides a non-transitory computer readable storage medium storing computer software code which when executed on a processor performs the method described above.

A further aspect provides a control system for an analytical instrument such as a mass spectrometer, the control system configured to cause the analytical instrument to perform the method described above.

A further aspect provides an analytical instrument, such as a mass spectrometer, comprising the control system described above.

The analytical instrument may comprise a first ion source, wherein the first ion source is an electron impact (EI) ionisation ion source, a direct laser ionisation ion source, or a field desorption ionisation ion source. The analytical instrument may comprise a second different ion source, which may comprise an electrospray (ESI) ion source or a chemical ionisation (CI) ion source.

The analytical instrument may comprise a mass analyser arranged downstream of the ion source(s). The mass analyser has a mass accuracy ≤about 100 ppm and a resolving power ≥about 10,000. The mass analyser may be an electrostatic orbital ion trap mass analyser, a Time-of-Flight (ToF) mass analyser, a multi-reflection Time-of-Flight (mrTOF) mass analyser, or a Fourier transform ion cyclotron resonance (FT-ICR) mass analyser. The analytical instrument may comprise a second different mass analyser, which can be any type of mass analyser such as an electrostatic orbital ion trap mass analyser, a Time-of-Flight (ToF) mass analyser, a multi-reflection Time-of-Flight (mrTOF) mass analyser, a Fourier transform ion cyclotron resonance (FT-ICR) mass analyser, an ion trap mass analyser or a quadrupole mass analyser.

The analytical instrument may comprise a mass filter arranged downstream of the ion source and upstream of the mass analyser. The mass filter may be as a quadrupole mass filter.

The analytical instrument may comprise a chromatographic separation device coupled to the ion source. The chromatographic separation device may be a liquid chromatography (LC) separation device or a gas chromatography (GC) separation device. The sample may be provided to the ion source(s) from the chromatographic separation device.

A second aspect provides a method of determining the presence of a compound in a sample, the method comprising:
generating sample ions from a sample by: ionising the sample using electrospray ionisation (ESI) or chemical ionisation (CI) to produce negatively charged parent ions, wherein the electrospray ionisation (ESI) or chemical ionisation (CI) is operated in negative ionisation mode, and fragmenting the parent ions using electron induced dissociation (EID) to produce the sample ions, wherein the sample ions include at least one positively charged fragment ion of interest that is indicative of the presence of the compound in the sample;
mass analysing the sample ions using a mass analyser so as to produce mass spectral data;
determining whether, in the mass spectral data, an ion peak is present that corresponds to the positively charged fragment ion of interest; and
when it is determined that an ion peak is present that corresponds to the positively charged fragment ion of interest: determining that the compound is present in the sample.

This aspect can, and in embodiments does, include any one or more or each of the optional features described herein. Thus, for example, the mass analyser can be configured as described above and elsewhere herein.

In this aspect, the compound may be a nonpolar compound, such as for example: a lipid, a hydrocarbon, or a perfluoro or polyfluoro compound (PFC) such as a perfluoroalkyl or polyfluoroalkyl substance (PFAS). The removal of an electron with excess energy from the negatively charged parent ion during electron induced dissociation (EID) results in a negatively charged fragment ion together with the characteristic positively charged fragment ion.

### DESCRIPTION OF THE DRAWINGS

Various embodiments will now be described in more detail with reference to the accompanying Figures, in which:

FIG. 1 shows a mass spectrum of a poly-fluorinated alcohol acquired with an Orbitrap™ instrument coupled to a gas chromatography (GC) interface;

FIG. 2 illustrates the detection of the CF3 cation from a GC separated poly-fluorinated alcohol using an Orbitrap™ instrument;

FIG. 3 illustrates schematically an analytical instrument configured in accordance with embodiments;

FIG. 4 illustrates schematically an analytical instrument configured in accordance with embodiments;

FIG. 5 illustrates schematically an analytical instrument configured in accordance with embodiments;

FIG. 6 illustrates schematically an analytical instrument configured in accordance with embodiments;

FIG. 7 illustrates schematically an analytical instrument configured in accordance with embodiments;

FIG. 8 illustrates schematically a method in accordance with embodiments; and

FIG. 9 illustrates schematically soft ionisation and electron impact dissociation (EID) of a PFAS compound Perfluorononanoic acid (PFNA).

### DETAILED DESCRIPTION

Embodiments provide a screening method for per- and polyfluoro compounds (PFCs), such as per- and polyfluoroalkyl substances (PFAS). This general screening tool can detect low abundances of PFAS or PFC in the environment such as in ground water or soil.

Until now, a general mass spectrometry-based screening method for PFCs has not been available. Prior art mass spectrometric methods typically use a targeted identification approach utilising a list of known PFAS or PFC compounds. Such targeted approaches have difficulty in identifying unknown PFCs or new types of synthesized substances such as perfluorated polyether. Previously unknown PFCs have a high probability of not being detected in such target-specific identification approaches.

PFAS and PFC have high hydrophobicity, and have a low probability of efficient ionisation by commonly applied soft ionisation techniques such as electrospray ionisation (ESI) and matrix-assisted laser desorption ionisation (MALDI). Consequently, such soft ionisation techniques provide relatively low sensitivity for PFAS and PFC compounds. A general ionisation process favouring ionisation of the hydrophobic tail and the high electron density of the perfluoro alkyl chain is more sensitive compared to the standard “soft” ESI.

It can be expected that the list of per- or poly-fluorinated substances (PFCs) will grow further in the future in response to the current and future knowledge of the environmental risks and toxicity of some PFAS and PFC compounds. Such new classes of substances include e.g., perfluorinated ethers. Although PFCs cannot be discovered by targeting perfluorinated alkyl substances, these new classes can be predicted to consistently share the presence of a hydrophobic molecular tail represented by the following two possible formulas CF2CF3 (fluorinated ethyl) or CF3 (fluorinated methyl).

Electron impact (EI) ionisation is an ionisation method in which energetic electrons interact with solid or gas phase atoms to produce electron deficient ions. This ionisation technique is considered to be a hard or harsh ionisation method as the typical energy of 70 eV exceeds that required to enable ionisation induced fragmentation. EI ionisation is widely used for different organic molecule classes. In contrast to softer ionisation techniques, EI ionisation typically leads to extensive fragmentation which is known to contribute to the compound-specific libraries for EI ionisation mass spectrometry.

To date, the presence of PFAS or PFC specific electron impact ionisation fragments has not been applied to screening for the presence of poly- or perfluorinated compounds in a mass spectrometric approach.

Embodiments provide a method for accurate screening of poly-fluorinated or per-fluorinated compounds (PFCs) that comprises using “harsh” electron impact (EI) ionisation to ionise a sample, and monitoring for the presence of ions with m/z=68.99466 at 100 ppm or better mass accuracy.

The CF3 fragment consists of three fluorine atoms and one carbon atom, and has a positive charge due to the electron deficient nature of the cation. In comparison to the relatively high molecular weight of per-fluorinated or poly-fluorinated compounds, the CF3 cation itself is a relatively low mass fragment ion having a nominal mass-to-charge ratio of 69. Although such a fragment ion with m/z 69 is known to be generated under harsh ionisation conditions, the accurate mass identity of the CF3 cation itself has not previously been used to confirm the presence of poly- or perfluorinated compounds when a sample is ionised under harsh electron impact (EI) conditions.

The reason for the use of highly specific mass detection is partly due to the unique property of the fluorine atom that has an exact mass less than the nominal mass of m/z=19. Another benefit of selecting the CF3 fragment ion or more generally a fragment ion having one or more fluorine atom(s) is the relatively high abundance of the ¹⁹F fluorine isotope, because fluorine is (beside 20 other elements) known to be a mononuclidic element. Such a single nuclide will have a characteristic single atomic mass.

Thus, in embodiments, the accurate mass of 68.99466, which represents exclusively the presence of a CF3 cation, is utilised to exclude any false positive ions with the same nominal mass of m/z 69. Such false positive ions could be present while screening for the CF3 cation. A comparison of the accurate masses of CF3 and various false positive ions at m/z 69 is given in Table 1.

TABLE 1
Accurate monoisotopic positive mass-to-charge
ratios of four different chemical compounds
sharing the nominal mass-to-charge ratio of 69

| | Monoisotopic mass | delta m/z * 1000 | ppm |
|---|---|---|---|
| CF3 | 68.9946612 | 0 | 0 |
| PF2 | 68.9700195 | −24.6417 | −357.2 |
| C3H4N2H | 69.00472 | 10.0588 | 145.8 |
| C3H3NO | 69.02092 | 26.2588 | 380.6 |

As can be seen from Table 1, the difference between the accurate monoisotopic mass of the CF3 cation and that of the three potential false positive positively charged fragment ions is higher than 100 ppm. Thus, with a highly accurate and highly resolving mass analyser, the resulting mass spectrum can be used to differentiate between the presence of the CF3 cation and other ions sharing the same nominal mass.

Electron impact (EI) ionisation of PFAS provides the benefit of higher specificity of ionisation of the alkyl chain, as the interaction of the electron with the higher electron density of the perfluorinated tail results in a higher probability of hydrophobic chain ionisation. In contrast, “softer” ionisation techniques (such as ESI or MALDI) favour the ionisation of more hydrophilic parts of PFAS. Thus, if no hydrophilic functional groups are present in the analysed PFAS compound, such substances are unlikely to be detected by the softer ionisation techniques mentioned above.

It will accordingly be understood that embodiments provide a method of screening for PFAS and PFC by a mass spectrometric data-independent approach utilising electron impact (EI) ionisation. Electron impact (EI) ionisation in positive ion polarity mode results in electron deficient radical cations that typically undergo fast cleavages. Accordingly, EI mass spectra of PFAS and PFC compounds show a high abundance of the CF3 cation at a nominal mass of m/z 69. Due to the mass deficient nature of the monoisotopic mass of the fluorine atom, the exact mass of the CF3 cation is slightly below the nominal mass of 69, namely at m/z=68.99466.

In reference EI mass spectra of perfluorooctanoic acid (PFOA, CAS registry number 335-67-1) the intact mass of m/z 414 representing the formula C8HF15O2 is not observable. The highest MS signal at m/z 395 shows the loss of fluorine atom from the intact mass. The signal at m/z 69 has a relative abundance of about 55%. This signal is within the top three highest signal abundance for the reference spectra of PFOA.

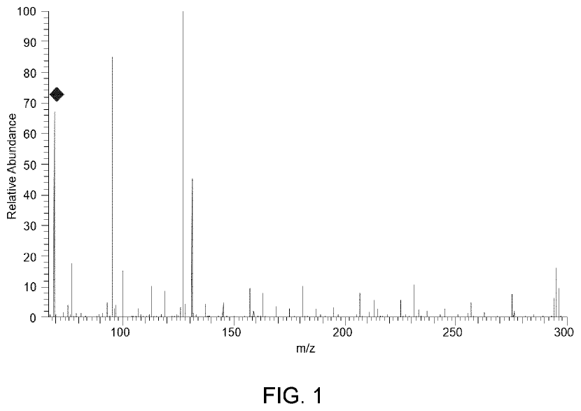

FIG. 1 shows an EI mass spectrum of a randomly chosen poly-fluorinated alcohol acquired with an Orbitrap™ Exploris™ 240 instrument coupled to a gas chromatography (GC) interface. The diamond indicates the signal corresponding to the CF3 cation having the calculated exact mass of m/z 68.99466.

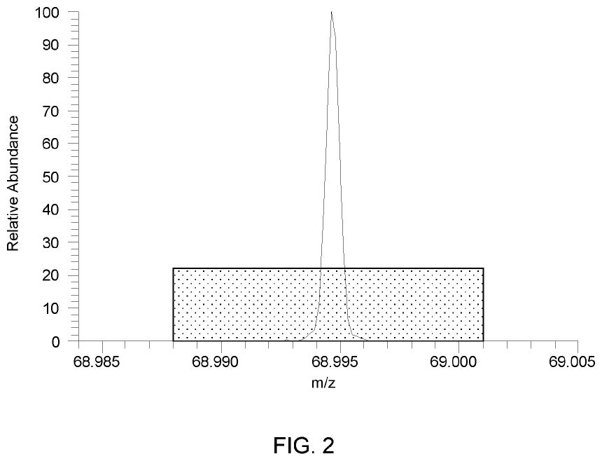

Accurate mass detection of the CF3 cation is shown in FIG. 2. To better visualise the confirmed presence of CF3, a zoomed-in mass range of m/z=68.98400-69.00500 is shown. In FIG. 2, the dotted box covers the mass range of 68.988-69.001, representing an accurate mass window of the expected theoretical CF3 cation mass-to-charge plus/minus the accurate detection window of 100 ppm for untargeted screening the presence of the CF3 cation.

The CF3 cation shown in FIG. 2 was acquired using a resolving setting of 60,000 at m/z 200 corresponding to a resolving power of 102,000 for the CF3 cation. The CF3 cation acquired in the mass spectrum shown matches the accurate mass of the calculated monoisotopic mass of 68.99466. The mass spectrum of the poly-fluorinated alcohol shown in FIGS. 1 and 2 was performed in a single scan having a measured signal-to-noise ratio of S/N=2.58e3.

Various practical implementations of the highly accurate screening method for PFAS or PFC compounds are possible.

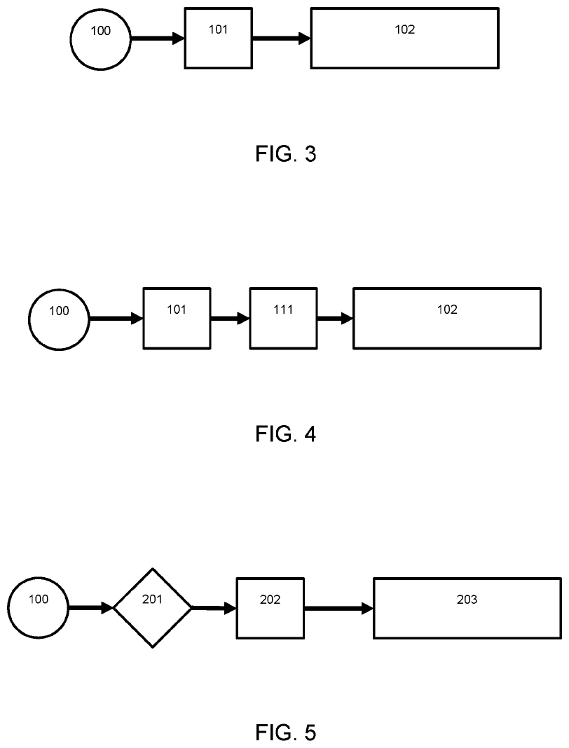

FIG. 3 illustrates a first embodiment, in which direct screening of a sample 100 is performed using harsh EI ionisation in an ionisation device 101, followed by highly accurate and highly resolved mass detection in an MS detector 102 of at least one compound specific fragment mass-to-charge ratio to confirm the presences of a per- or poly-fluorinated compound.

FIG. 4 illustrates a second embodiment in which direct screening of a sample 100 is performed using harsh EI ionisation in an ionisation device 101, followed by the isolation of a compound-specific fragment ion in an isolation device 111 such as a quadrupole mass filter, and then highly accurate and highly resolved mass detection in an MS detector 102 of at least one compound specific fragment mass-to-charge ratio to confirm the presences of a per- or poly-fluorinated compound.

FIG. 5 illustrates a further embodiment which includes chromatographic or another type of applicable separation or purification by a separation or purification device 201 of a sample 100, followed by harsh EI ionisation in an ionisation device 202, and highly accurate and highly resolved mass detection in an MS detector 203 of at least one compound specific fragment mass-to-charge ratio to confirm the presences of a per- or poly-fluorinated compound.

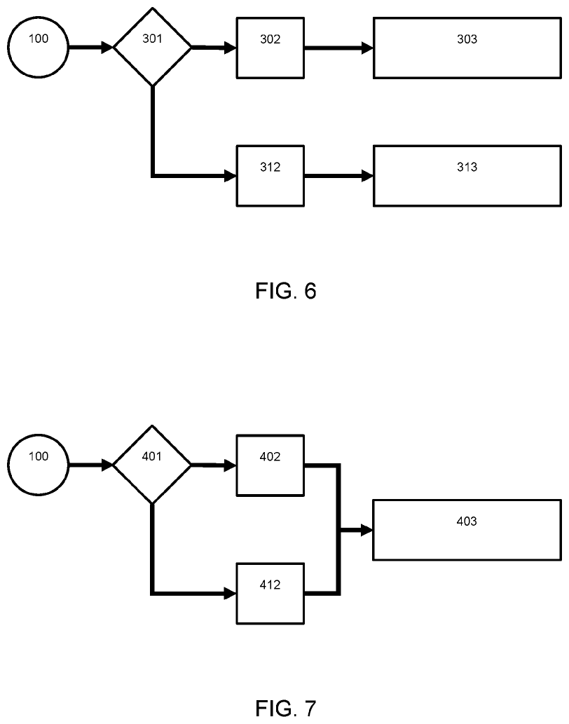

FIG. 6 illustrates a further embodiment which includes chromatographic or another type of applicable separation or purification by a separation or purification device 301 of a sample 100, followed by splitting the sample to be simultaneously analysed under both harsh and soft ionisation conditions by at least two different ionisation devices 302 and 312 connected to two different mass analysers 303 and 313. Soft ionisation of the sample in the soft ionisation source 312 will prefer ionisation of an intact poly-fluorinated or perfluorinated molecule. The retention time of the confirmed presence of a per- or poly-fluorinated compound in the mass detector 303 is utilised to target the identification of the poly-fluorinated or perfluorinated compound. In these embodiments, the two types of ionisation may be performed simultaneously during the same chromatographic run either by continuously splitting the chromatographic eluent between the two sources or by rapidly switching the chromatographic eluent between the two ion sources.

FIG. 7 illustrates a further embodiment which includes chromatographic or another type of applicable separation or purification by a separation or purification device 401 of a sample 100, followed by splitting the sample to be simultaneously analysed under both harsh and soft conditions by at least two different ionisation devices 402 and 412. Both ionisation devices 402 and 412 are connected to a to a single mass analyser 403. Again, the two types of ionisation may be performed simultaneously during the same chromatographic run either by continuously splitting the chromatographic eluent between the two sources or by rapidly switching the chromatographic eluent between the two ion sources. Alternatively, the harsh and soft ionisations and detection may be performed in two separate (e.g. sequential) chromatographic runs. In these embodiments, detection of ions from both ion sources may be performed by continuously rapidly switching between the two ion sources.

It will be understood from the above that the method of non-targeted screening for poly- or perfluorinated compounds with a perfluorinated CF3-tail may include the following steps:
1. Harsh electron impact (EI) ionisation of a sample under investigation as possibly including a poly- or perfluorinated compound; and
2. Unambiguous detection of a harsh ionisation fragment, such as in particular electron impact ionisation resulting in at least one known perfluorinated fragment ion; where
   a. The unambiguous identification covers the signal abundance of at least one CF3-specific poly- and/or perfluorinated fragment ion such as CF3 (m/z 68.99466), CF3CF2 (m/z 118.99147) or CF3CF═CF (130.99147) above significant S/N threshold, e.g. higher than 50;
   b. The unambiguous identification is achieved by accurate mass detection within a predefined accurate mass window of the expected accurate mass plus/minus a mass accuracy of less than around 100 ppm; and
   c. The resolving power of the mass detecting device is high enough to ensure the unambiguous identification of the presence of the fragment ion.

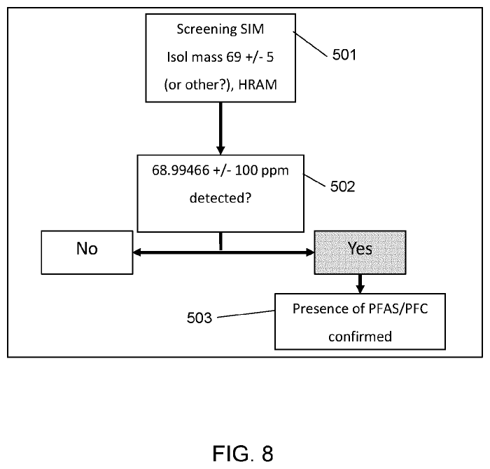

FIG. 8 illustrates a screening method according to an embodiment, in which a selected ion monitoring (SIM) screening window at m/z 69±5 is used when mass analysing a sample using a high-resolution accurate-mass (HRAM) instrument (step 501). The resulting mass spectrum is analysed to determine whether an ion peak at m/z 68.99466±100 ppm has been detected (step 502). If not, then a PFAS/PFC compound has not been detected in the sample, but if the ion peak is present then the presence of a PFAS/PFC compound in the sample is confirmed (step 503).

As is also described above, in addition to the unambiguous screening for the presence of known EI-induced fragment ions, a chromatographic separation of the sample can be used to assist further compound specific identification of the per- or poly-fluorinated compound, where the chromatographically separated sample is independently ionised by a soft ionisation technique such as, e.g., electrospray ionisation (ESI) or chemical ionisation (CI). Such separation can be executed in parallel to the electron impact (EI) ionisation of the separated sample or afterwards if the retention time of the eluent can be reproduced.

Thus, in some embodiments, the screening method is combined with a chromatographic separation such as gas chromatography (GC) or liquid chromatography (LC) or another fluidic based separation device, and the reproducible retention time attribute is utilised to further confirm the substance's identity.

Embodiments provide non-targeted screening method for poly- and/or perfluorinated compounds e.g. from environmental samples. The screening method which includes EI-ionisation is highly sensitive and highly PFC- and/or PFAS-specific. The selection of compound specific fragment ions can be tailored for screening of sub-classes of PFAS and PFC, if further class specific EI-fragment ions are selected.

In some embodiments, a known concentration of a C13 enriched isotopically labelled sample is added to the analysed sample, and the corresponding relative intensities between fragment ions of the sample and the isotopically enriched sample is used to internally determine the concentration of the PFC or PFAS.

The highly accurate and high mass resolving screening method for compound-specific fragment ions such as m/z 68.99466 specifically representing the CF3 cation is applicable to highly accurate mass spectrometric instruments such as orthogonal time of flight (TOF) instruments, Orbitrap™ instruments, FT ICR instruments and multi-reflection time of flight (mrTOF) instruments. For less accurate mass determining instruments or instruments having a lower resolving power (e.g. typically providing nominal mass detection) such as quadrupoles or ion traps, identification of false positive ions cannot be excluded. Thus, embodiments may use any type of highly accurate mass analyser such as e.g. an Orbitrap™ mass analyser, TOF analyser, mrTOF analyser or FT ICR analyser, offering at least the specified resolution to uniquely confirm the presence of the compound specific fragment ion.

In particular, the resolving power at mass-to-charge ratio 69 should be higher than about 50% of the +/−100 ppm window to allow positive identification when screening for the CF3 cation at m/z 68.99466. The corresponding FWHM is (68.988-69.001)/2=0.0069. This minimum FWHM value corresponds to a resolving power of 10,000. The presence of ions having a nominal mass of m/z 69 itself is indicative of ions other than the compound-specific CF3 cation. Therefore, detection of ions with a nominal mass of 69 alone is not specific enough for screening of poly- or perfluorinated compounds.

Although the CF3 cation is particularly useful in the methods described herein, other poly- or perfluorinated specific fragment ions having higher mass-to-charge ratios than m/z 68.99466 can be included, e.g. in a list of specific screening candidates.

Although electron impact ionisation is particularly suitable to the methods described herein, other harsh ionisation techniques such as direct laser ionisation and field desorption ionisation can provide similar ionisation of non-polar compounds and compound activation yielding fragmentation.

Alternatively, a soft ionisation technique such as electrospray ionisation (ESI) or chemical ionisation (CI) may be used together with electron induced dissociation (EID) to produce ions universally indicative of the presence of a PFAS compound, e.g. the CF3 cation. In particular embodiments, an electrospray ionisation (ESI) source is configured to ionise the eluent from a liquid chromatography (LC) separation device, and the so-produced ions are subjected to EID.

To ionise PFAS compounds with ESI, the more sensitive electrospray polarity is the negative ion polarity (based on the property of the functional chemical group being a carbonic acid, sulfonamido acid or sulfonic acid). The Perfluoro-alkyl “tail” is not directly involved in the ESI ionisation of PFAS. Ionising the PFAS in positive ESI is possible but it is less sensitive.

Thus, embodiments use soft ionisation of the compounds in negative polarity and then electron impact of the PFAS tail to generate the CF3 (or analog) longer CₘFₘ positively charged tails, i.e. a “charge inversion”.

A more general formula for PFAS having a carbocyclic acid representing the head of an amphiphilic compound is the following:

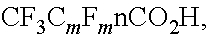

where m and n are integer values. Soft ionisation (e.g. ESI) in negative polarity provides the PFAS anion:

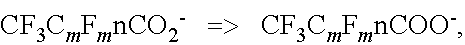

and electron impact dissociation (EID) of the PFAS anion provides:

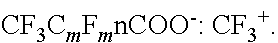

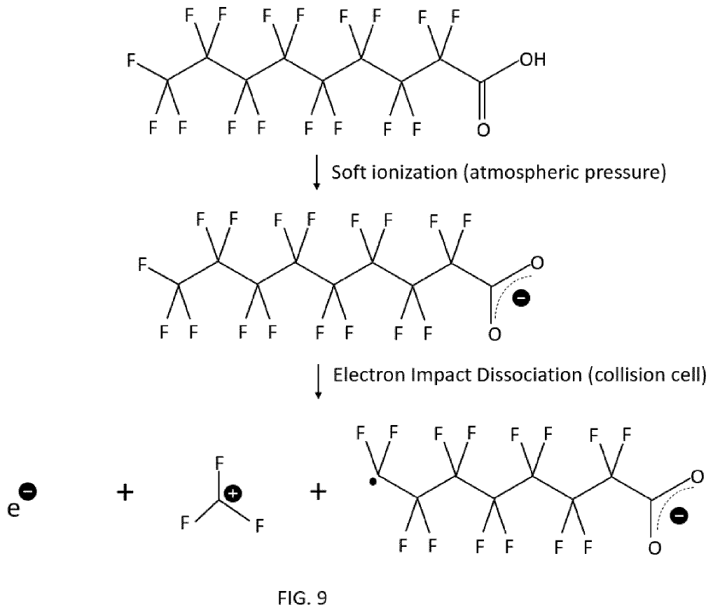

FIG. 9 illustrates soft ionisation of Perfluorononanoic acid generating a negatively charged ion at m/z 463. Electron impact ionisation of the amphiphilic compound results in the generation of (i) a negatively charged hydrophilic fragment and (ii) the positively charged perfluorated cation CF3 which is itself indicative of the presence of PFAS.

In cases where the CF3 cation is not detectable (e.g. where the CF3 cation is not trapped), the accurate loss of the mass of the CF3 cation itself can be utilised to confirm the presence of the perfluorated tail.

It is believed that this “charge inversion” process is new and advantageous in its own right and is applicable to compounds other than PFAS compounds. Advantageously, this “charge inversion” process allows for the production of characteristic positively charged fragment ions from certain compounds even while using electrospray ionisation (ESI) operating in its more sensitive negative ionisation mode. Thus, a second aspect is provided, as described above.

In general, the compound may be a nonpolar compound, such as for example: a lipid, a hydrocarbon, or a perfluoro or polyfluoro compound (PFC) such as a perfluoroalkyl or polyfluoroalkyl substance (PFAS). The removal of an electron from the negatively charged parent ion during electron induced dissociation (EID) results in a negatively charged fragment ion together with the characteristic positively charged fragment ion.

The negatively charged parent ion can be trapped simultaneously with the positively charged fragment ion of interest in a fragmentation device, e.g. by a quadrupolar trapping potential via radio frequency.

Although the method may be applied to the analysis of PFAS compounds, this “charge inversion” can be applied to other amphiphilic ionic species as long as the negatively charged head of the molecule does not take part of the electron impact and is therefore untouched and only the more hydrophobic tail interacts with the electron impacting the molecule.

Although the present invention has been described with reference to various embodiments, it will be understood that various changes may be made without departing from the scope of the invention as set out in the accompanying claims.

1. A method of determining the presence of a perfluoroalkyl or polyfluoroalkyl substance (PFAS) in a sample, the method comprising:
generating sample ions from a sample either: (i) by using electron impact (EI) ionisation, direct laser ionisation, or field desorption ionisation; or (ii) by using electrospray ionisation (ESI) or chemical ionisation (CI), and electron induced dissociation (EID);
mass analysing the sample ions using a mass analyser so as to produce mass spectral data, wherein the mass analyser has a mass accuracy ≤about 100 ppm and a resolving power ≥about 10,000;
determining whether, in the mass spectral data, an ion peak is present that has an accurate mass to charge ratio within ±100 ppm of a first accurate mass to charge ratio m/z, wherein the first accurate mass to charge ratio m/z is the accurate mass to charge ratio of an ion indicative of the presence of a PFAS in the sample; and
when it is determined that an ion peak is present that has an accurate mass to charge ratio within ±100 ppm of the first accurate mass to charge ratio m/z: determining that a PFAS is present in the sample;
wherein:
the ion indicative of the presence of a PFAS is a CF3 cation having a nominal mass to charge ratio of 69, and the first accurate mass to charge ratio m/z=68.99466; and/or
the ion indicative of the presence of a PFAS is a CF3CF2 cation having a nominal mass to charge ratio of 119, and the first accurate mass to charge ratio m/z=118.99147; and/or
the ion indicative of the presence of a PFAS is a CF3CF cation having a nominal mass to charge ratio of 131, and the first accurate mass to charge ratio m/z=130.99147.

2. The method of claim 1, wherein the sample ions are generated from the sample using electron impact (EI) ionisation.

3. The method of claim 1, wherein the mass analyser is an electrostatic orbital ion trap mass analyser, a Time-of-Flight (ToF) mass analyser, a multi-reflection Time-of-Flight (mrTOF) mass analyser, or a Fourier transform ion cyclotron resonance (FT-ICR) mass analyser.

4. The method of claim 3, wherein the mass analyser is an electrostatic orbital ion trap mass analyser.

5. The method of claim 1, further comprising, before the step of mass analysing the sample ions: isolating sample ions having mass to charge ratios within a mass to charge ratio window centred at the nominal mass to charge ratio of the ion indicative of the presence of a PFAS.

6. The method of claim 5, wherein the step of isolating sample ions comprises isolating sample ions using a mass filter.

7. The method of claim 5, wherein the mass to charge ratio window has a width ≤10 m/z, or ≤5 m/z.

8. The method of claim 5, wherein:
the ion indicative of the presence of a PFAS is a CF3 cation, and the mass to charge ratio window is centred at a mass to charge ratio of 69; and/or
the ion indicative of the presence of a PFAS is a CF3CF2 cation, and the mass to charge ratio window is centred at a mass to charge ratio of 119; and/or
the ion indicative of the presence of a PFAS is a CF3CF cation, and the mass to charge ratio window is centred at a mass to charge ratio of 131.

9. The method of claim 1, wherein the step of determining whether an ion peak is present comprises determining whether an ion peak is present that has an intensity above a signal to noise ratio threshold.

10. The method of claim 1, further comprising chromatographically separating the sample before the step of generating sample ions from the sample.

11. The method of claim 10, wherein the step of chromatographically separating the sample comprises separating the sample using liquid chromatography (LC) or gas chromatography (GC).

12. The method of claim 10, further comprising:
splitting the chromatographically separated sample into a first part and a second part, wherein the sample ions are first sample ions generated from the first part of the sample using a first ion source, wherein the mass spectral data is first mass spectral data, and wherein the method further comprises:
generating second sample ions from the second part of the sample using a second different ion source; and
mass analysing the second sample ions so as to produce second mass spectral data.

13. The method of claim 12, wherein the second ion source is an electrospray ionization (ESI) ion source or a chemical ionisation (CI) ion source.

14. The method of claim 12, wherein:
the first sample ions and the second sample ions are mass analysed using the same mass analyser; or
the first sample ions are mass analysed using a first mass analyser, and the second sample ions are mass analysed using a second different mass analyser.

15. The method of claim 12, further comprising:
when it is determined that an ion peak is present that has an accurate mass to charge ratio within ±100 ppm of the first accurate mass to charge ratio m/z: determining a chromatographic retention time associated with the ion peak; and
characterising and/or identifying the PFAS present in the sample using the second mass spectral data associated with the determined chromatographic retention time.

16. The method of claim 1, further comprising: when it is determined that an ion peak that has an accurate mass to charge ratio within ±100 ppm of the first accurate mass to charge ratio m/z is not present in the mass spectral data: determining that a PFAS is not present in the sample.

17. The method of claim 1, wherein the sample comprises a known concentration of labelled sample, wherein the sample ions comprise sample fragment ions and labelled sample fragment ions, and wherein the method further comprises:
comparing intensities of the sample fragment ions to intensities of the labelled sample fragment ions; and
determining the concentration of PFAS present in the sample based on the comparison(s).

18. The method of claim 17, wherein the labelled sample comprises C13 enriched labelled sample.

19. A non-transitory computer readable storage medium storing computer software code which when executed on a processor performs the method of claim 1.

20. A control system for an analytical instrument, the control system configured to cause the analytical instrument to perform the method of claim 1.

21. An analytical instrument, comprising a control system configured to cause the analytical instrument to perform the method of claim 1.
